(19) United States
(12) Patent Application Publication
(10) Pub. No.: US 2010/0151036 A1
(43) Pub. Date: Jun. 17, 2010

(54) Multiphase drug delivery system

(76) Inventors: Bin Wu, Sharon, MA (US); Yanbin Huang, Beijing (CN)

Correspondence Address: Bin Wu, 80 Bullard St., Sharon, MA, 02067, US

(21) Appl. No.: 12/316,659

(22) Filed: Dec. 16, 2008

(51) Int. Cl. A61K 9/14; A61K 47/30; A61P 43/00

(52) U.S. Cl. 424/499; 424/489; 424/501; 514/772.3

(57) ABSTRACT

A two-phase drug delivery medium comprising a discontinuous phase and a solid continuous phase, the discontinuous phase comprising a plurality of droplets, each of which comprises a fluid and at least one drug dissolved or suspended within the fluid, and the continuous phase surrounding and encapsulating the discontinuous phase.

### CROSS-REFERENCE TO RELATED APPLICATIONS

This application claims priority to U.S. Provisional Applications 61/007,432 (filed Dec. 13, 2007).

### FIELD OF THE INVENTION

This invention relates to a two-phase drug delivery medium and methods for the preparation thereof. This invention further relates to the use of such drug delivery medium in pharmaceutical compositions and methods of treating mammals. More specifically, this invention relates to a two-phase drug delivery medium which comprises a discontinuous phase containing drug solutions or drug particles and a continuous phase surrounding and encapsulating the discontinuous phase. The present application also describes materials useful in fabricating such media.

### BACKGROUND OF THE INVENTION

Bioavailability is the degree to which a drug becomes available to its site of action after administration. For example, many factors such as in vivo dissolution, absorption, and metabolism can affect the bioavailability of orally administered drugs. Due to the intrinsic limit of currently used drug discovery technologies, more and more newly discovered drug candidates have poor water solubility, and this has become a significant problem in the pharmaceutical development. Poorly water soluble drugs, i.e., those having dose/water solubility ratio larger than 250 mL (the “FDA glass of water”), tend to leave significant amount unabsorbed during the gastrointestinal tract transit, and hence cause erratic absorption and/or high inter-individual variation in absorption. Therefore, there have been intensive R&D efforts both in the pharmaceutical industry and in academia to develop drug delivery technologies for these poorly-soluble drugs.

Currently, several technologies are used for the delivery of poorly-water soluble drugs. One of these technologies is nanosizing, which utilizes media milling, nano-precipitation, and homogenization to convert therapeutic compounds to particles having sizes of below 1 micron. These nanosized particles can further be stabilized in a water-based stabilizer solution. The solubility of the drug is enhanced due to reduced particle sizes and increased particle surface areas. Exemplary methods of nanosizing are described in U.S. Pat. No. 5,145,684 for “Surface Modified Drug Nanoparticles;” U.S. Pat. No. 5,518,187 for “Method of Grinding Pharmaceutical Substances;” U.S. Pat. No. 5,156,842 for “Liquid Suspension for Oral Administration;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” U.S. Pat. No. 5,470,583 for “Method of Preparing Nanoparticle Compositions Containing Charged Phospholipids to Reduce Aggregation;” and U.S. Pat. No. 7,390,505 for “Nanoparticulate Topiramate Formulations.”

Another method currently used for delivery of poorly water-soluble drugs is solid solution or dispersion technology. Essentially, a drug is dissolved or dispersed in a solid polymer matrix. For example, Abbott's Meltrex™ (Zhu, T, et al, 45^{th} Interscience Conference on Antimicrobial Agents and Chemotherapy, Washington, D.C., Dec. 16-19, 2005) process provides a method of enhancing bioavailability by forming a polymer-drug matrix. In this process, the drug substance itself together with a pharmaceutically-acceptable polymer and additional excipients are fed continuously through a twin-screw extruder. The ingredients are thoroughly mixed and the drug substance is present in the resulting matrix either as crystalline particles embedded in the polymer or as a solid dispersion actually dissolved in the polymer. Enhanced bioavailability is achieved by the formation of the solid dispersion. The presence of the drug in this solid molecular dispersed state has been shown to increase significantly the absorption of poorly soluble compounds from the gastro-intestinal tract.

Lipid based drug delivery formulations generally consist of a drug dissolved in a blend of two or more excipients, which may be triglyceride oils, partial glycerides, surfactants or co-surfactants. This technology leads to improved bioavailability because the slow dissolution process which limits the bioavailability of poorly water soluble drugs can be avoided. The formulation allows the drug to remain in a dissolved state throughout its transit through the gastrointestinal tract. The availability of the drug for absorption can be enhanced by presentation of the drug as a solubilizate within a colloidal dispersion. The bioavailability enhancement can be achieved by formulation of the drug in a self-emulsifying system or alternatively by taking advantage of the natural process of triglyceride digestion. In practice ‘lipid’ formulations range from pure oils to blends which contain a substantial proportion of hydrophilic surfactants or co-solvents. A number of authors have overviewed the lipid based drug delivery systems. For example, Jannin, V. et al in “Approaches for the development of solid and semi-solid lipid-based formulations,” Advanced Drug Delivery Reviews, 60 (2008), 734-746; Bally, M. B., et al in “Controlling the Drug Delivery Attributes of Lipid-Based Formulations,” Journal of Liposome Research, 1998, and Pouton, C. W. in “Lipid formulations for oral administration of drugs: non-emulsifying, self-emulsifying and ‘self-microemulsifying’ drug delivery systems,” European Journal of Pharmaceutical Sciences, Volume 11, Supplement 2, October 2000, Pages S93-S98.

Technology involving oil-in-water emulsions such as macroemulsion, miniemulsion, microemulsion, and nanoemulsion is also used to enhance solubility and bioavailability of poorly water soluble drugs. For example, U.S. Pat. No. 6,638,537 for “Microemulsion and Micelle Systems for Solubilizing Drugs” describes a microemulsion delivery system for water insoluble or sparingly water soluble drugs that comprises a long polymer chain surfactant component and a short fatty acid surfactant component, with the amount of each being selected to provide stable microemulsion or micellar systems. Other examples include U.S. Pat. No. 7,205,279 for “Pharmaceutical Compositions;” U.S. Pat. No. 7,153,525 for “Microemulsions as precursors for nanoparticles;” U.S. Pat. No. 7,115,565 for “Chemotherapeutic microemulsion compositions of paclitaxel with improved oral bioavailability;” U.S. Pat. No. 6,716,801 for “Compositions and methods for their preparation,” and the references cited therein.

Each of the technology mentioned above has its own advantages and has been used in commercial products. However, they also have serious limitations which prevent them from universal applications. For example, in the nanosizing method, liquid suspension is first generated and a drying step is required to make desirable solid dosage forms, but it is often difficult to re-disperse the resulting nanoparticles in vivo without significant size change. The method of drug-polymer solid dispersion is a seemingly simple process, but it consists of many shortcomings. Specifically, these include limited drug loading capacity, poor stability of blend morphology, difficulty achieving reproducibility of physico-chemical properties upon scale-up, instability during manufacturing and storage, in vivo re-precipitation, and the necessity for high processing temperatures when melt processing. Lipid-bases systems can be used in solid dosage forms only when solid lipid is used, which seriously limits its application.

Therefore, there is unmet need to develop new technologies to increase the bioavailability of poorly water soluble drugs that is compatible with various dosage forms.

### SUMMARY OF THE INVENTION

Accordingly, this invention provides a two-phase drug delivery medium comprising a discontinuous phase and a continuous phase, the discontinuous phase comprising a plurality of droplets, each of which comprises a fluid and at least one drug dissolved or suspended within the fluid, and the continuous phase surrounding and encapsulating the discontinuous phase.

This invention also provides a process for producing a two-phase drug delivery medium, this process comprising:

providing a liquid medium comprising a solid-forming material;

dispersing in the liquid medium a plurality of droplets, each of which comprises a fluid and at least one drug dissolved or dispersed within the fluid; and

subjecting the liquid medium to conditions effective to cause the solid-forming therein to form solid or semi-solid, and thereby producing a two-phase drug-delivery medium in which the solid-forming material forms a continuous phase surrounding and encapsulating the droplets, which form the discontinuous phase of the drug delivery medium.

### BRIEF DESCRIPTION OF DRAWINGS

Preferred embodiments of the invention will now be described, though by ways of illustration only, with reference to the accompanying drawings, in which:

FIG. 1 depicts the two phase drug delivery medium of the current invention, wherein the drug is dissolved in the discontinuous phase fluid, which forms the droplets.

FIG. 2 depicts the two phase drug delivery medium of the current invention, wherein the drug particles are suspended in the discontinuous phase fluid, which forms the droplets.

FIG. 3 shows schematically regularly and irregularly shaped droplets.

### DETAILED DESCRIPTION OF THE INVENTION

A. Definitions

The following definitions apply.

The term “nanodroplets” refer to a group of small, spherically or non-spherically shaped particles of a liquid suspended in a medium, where the mean value of the diameter or quasi-diameter (as defined below) of the particles within the group is below about 1 micron.

The term “microdroplets” refer to a group of small, generally spherically or non-spherically shaped particles of a liquid suspended in a medium, where the mean value of the diameter or quasi-diameter (as defined below) of the particles within the group is between 1 to 1,000 microns.

The term “a quasi-diameter” applies to non-sphere shaped, i.e., not perfectly spherical droplets and refers to the length of the longest of a plurality of straight lines connecting the following three points: the geometrical center of a droplet, and two points on its surface.

The term “a lipid” refers to compounds of biological origin that are typically water-insoluble or non-polar, including aliphatic, cyclic and aromatic compounds generally classified as fatty acids, fatty-acid derived phospholipids, sphingolipids, glycolipide waxes, and terpenoids, such as retinoids and steroids.

The term “a therapeutically active compound” is used interchangeably with “a drug” and refers to a compound which, when administered to a mammal in need thereof, may elicit a beneficial therapeutic response.

The term “an emulsion” is defined as a colloid system in which both phases are liquids.

The term “a suspension” is defined as a colloid system that has a continuous liquid phase in which a solid is suspended.

The terms “a stable emulsion” and “a stable suspension” are defined as an emulsion or a suspension, respectively, in which the phases do not separate for a substantial period of time.

B. Embodiments of the Invention

As already mentioned, the present invention provides a two-phase drug delivery medium comprising a discontinuous phase and a continuous phase, the discontinuous phase comprising a plurality of droplets, each of which comprises a fluid and at least one drug dissolved or suspended within the fluid, and the continuous phase surrounding and encapsulating the discontinuous phase.

In the present drug delivery medium, the discontinuous phase may comprise from 0.1 to 95% by volume of the medium, but preferably comprises about 5 to 50% by volume.

The fluid of the droplets in the discontinuous phase is preferably a biologically compatible oil. A variety of biocompatible oils may be used for making the droplets in the discontinuous phase of the present invention.

In the following description it will be understood that the nature of the oils is not critical beyond those particular qualifications set forth below, and may generally be any such known materials conventionally employed and which are accepted in the food and pharmaceutical industry.

The oil, or mixtures thereof, may be liquid at room temperature, although in some cases, mild heating of a solid oil to form a liquid is acceptable.

A biocompatible oil capable of solubilizing the therapeutically active compound may be used.

The biocompatible oil may have a viscosity at ambient temperature ranging from 0.1 to 10,000 centipoise, preferably 1 to 5,000 centipoise, more preferably from 1 to about 500 centipoise. For example, triacetin, diacetin, tocopherol, or mineral oils may be used.

Other examples of biocompatible oils that may be used include such oils listed in U.S. Pat. No. 5,633,226, the disclosure of which is hereby incorporated in its entirety by reference herein, and include CAPTEX™ 200, WHITEPSOL™ H-15 and MYVACET™ 9-45K, hydrogenated cocoa oil, coconut oil, elm seed oil, palm oil, cottonseed oil, soybean oil, parsley seed oil, mustard seed oil, linseed oil, tung oil, pomegranite seed oil, laurel oil, rapeseed oil, corn oil, evening primrose oil, maize oil, olive oil, persic oil, poppy-seed oil, safflower oil, sesame oil, soya oil, sunflower oil, ethyl oleate oil, Japanese anise oil, oil of eucalyptus, rose oil, almond oil, arachis oil, castor oil, mineral oil, peanut oil, vegetable oil and derivatives, sucrose polyester, silicone oil, and paraffin oil.

The biocompatible oil may also be those that are solid or semi-solid at ambient temperatures but melt into liquid when heated. Examples of useful lipids are phospholipids, saturated and unsaturated fatty acids, lysolipids, and lipids carrying hydrophilic polymers. For example, dipalmitoylphosphatidylcholine (DPPC) or distearoylphosphatidylcholine (DSPC) may be used. Other examples of lipids that may be used include dioleoylphosphatidylcholine, dimyristoylphosphatidylcholine, dipentademayoylphosphatidylcholine, dilauroylphosphatidylcholine, dioleoylphosphatidylcholine, phosphatidylethanolamines (e.g., dioleoylphosphatidylethanolamine), phosphatidylserine, phosphatidylglycerol, phosphatidylinositol, sphingolipids (e.g., sphingomyelin), glycolipids (e.g., ganglioside GM1 and GM2), glucolipids, sulfatides, glycosphingolipids, phosphatidic acid, lipids bearing polymers (e.g., bearing polyethyleneglycol (“PEGylated lipids”), chitin, hyaluronic acid or polyvinylpyrrolidone), lipids bearing polysaccharides (e.g., bearing sulfonated mono-, di-, oligo- or polysaccharides), cholesterol, cholesterol sulfate, cholesterol hemisuccinate, tocopherol hemisuccinate, lipids with ether and ester-linked fatty acids, polymerized lipids, diacetyl phosphate, stearylamine, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3-.beta.-yloxy)-1-thio-.beta.-D-galactopyranoside, digalactosyldiglyceride, 6-(5-cholesten-3-.beta.-yloxy)hexyl-6-amino-6-deoxy-1-thio-.beta.-D-galac-to pyranoside, 6-(5-cholesten-3-.beta.-yloxy)hexyl-6-amino-6-deoxyl-1-thio-.alpha.-D-man-no pyranoside, 12-(((7′-diethylaminocoumarin-3-yl)carbonyl)methylamino)-octademayoic acid, N-[12-(((7′-diethylaminocoumarin-3-yl)carbonyl)methyl-amino)octadem-ayoyl]-2-aminopalmitic acid, cholesteryl(4′-trimethylammonio)butanoate, N-succinyldioleoylphosphatidylethanolamine, 1,2-dioleoyl-sn-glycerol, 1,2-dipalmitoyl-sn-3-succinylglycerol, 1,3-dipalmitoyl-2-succinylglycerol; 1-hexadecyl-2-palmitoylglycerophosphoethanolamine, palmitoylhomocysteine, and combinations thereof.

Additionally, PEGylated lipids that may be used include poly(ethylene glycol) (PEG)-based lipids having a molecular weight of between about 1,000 Daltons and 10,000 Daltons, for example about 2,000 Daltons, 5,000 Daltons, or 8,000 Daltons.

Saturated and unsaturated fatty acids that may be used include molecules that have between 12 carbon atoms and 22 carbon atoms in either linear or branched form. Some examples of specific saturated fatty acids that may be used include, but are not limited to, lauric, myristic, palmitic, and stearic acids. Some examples of specific unsaturated fatty acids that may be used include, but are not limited to, lauroleic, physeteric, myristoleic, palmitoleic, petroselinic, linoleic, and oleic acids. Some examples of specific branched fatty acids that may be used include, but are not limited to, isolauric, isomyristic, isopalmitic, and isostearic acids and isoprenoids.

A variety of therapeutically active compounds may be used for solubilization and bioavailability enhancement using the present invention. The specific nature of a therapeutically active compound to be used may be determined by the kind of disease or disorder that is intended to be treated. For example, anti-cancer agents may be used as therapeutically active compounds, e.g., TAXOL®. or paclitaxel, among other kinds of drugs.

Other therapeutically active compounds that may be used in the present invention include but are not limited to:

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);

anesthetics (e.g., halothane, isoflurane, methoxyflurane, propofol, thiobarbiturates, xenon and the like);

antiasthmatics (e.g., Azelastine, Ketotifen, Traxanox, and the like);

antibiotics (e.g., neomycin, streptomycin, chloramphenicol, cephalosporin, ampicillin, penicillin, tetracycline, and the like);

antidepressants (e.g., nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, protriptyline hydrochloride, and the like);

antidiabetics (e.g., biguanides, hormones, sulfonylurea derivatives, and the like);

antifungal agents (e.g., griseofulvin, keoconazole, amphotericin B, Nystatin, candicidin, and the like);

antihypertensive agents (e.g., propanolol, propafenone, oxyprenolol, nifedipine, reserpine, trimethaphan camsylate, phenoxybenzamine hydrochloride, pargyline hydrochloride, deserpidine, diazoxide, guanethidine monosulfate, minoxidil, rescinamine, sodium nitroprusside, rauwolfia serpentina, alseroxylon, phentolamine mesylate, reserpine, and the like);

anti-inflammatories (e.g., (non-steroidal) indomethacin, naproxen, ibuprofen, ramifenazone, piroxicam, (steroidal) cortisone, dexamethasone, fluazacort, hydrocortisone, prednisolone, prednisone, and the like);

antineoplastics (e.g., adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, and the like);

antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene, and the like);

immunosuppressive agents (e.g., cyclosporine, azathioprine, mizoribine, FK506 (tacrolimus), and the like); antimigraine agents (e.g., ergotamine tartrate, propanolol hydrochloride, isometheptene mucate, dichloralphenazone, and the like);

sedatives/hypnotics (e.g., barbiturates (e.g., pentobarbital, pentobarbital sodium, secobarbital sodium), benzodiazapines (e.g., flurazepam hydrochloride, triazolam, tomazeparm, midazolam hydrochloride, and the like);

antianginal agents (e.g., beta-adrenergic blockers, calcium channel blockers (e.g., nifedipine, diltiazem hydrochloride, and the like), nitrates (e.g., nitroglycerin, isosorbide dinitrate, pentaerythritol tetranitrate, erythrityl tetranitrate, and the like));

antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like);

antimanic agents (e.g., lithium carbonate);

antiarrhythmics (e.g., amiodarone, related derivatives of amiodarone, bretylium tosylate, esmolol hydrochloride, verapamil hydrochloride, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, lidocaine hydrochloride, and the like);

antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium, and the like);

antigout agents (e.g., colchicine, allopurinol, and the like);

anticoagulants (e.g., heparin, heparin sodium, warfarin sodium, and the like);

thrombolytic agents (e.g., urokinase, streptokinase, altoplase, and the like);

antifibrinolytic agents (e.g., aminocaproic acid);

hemorheologic agents (e.g., pentoxifylline);

antiplatelet agents (e.g., aspirin, empiriri, ascriptin, and the like);

anticonvulsants (e.g., valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione, and the like);

antiparkinson agents (e.g., ethosuximide, and the like);

antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like);

agents useful for calcium regulation (e.g., calcitonin, parathyroid hormone, and the like);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and the like);

antiviral agents (e.g., interferon gamma, zidovudine, amantadine hydrochloride, ribavirin, acyclovir, and the like);

antimicrobials (e.g., cephalosporins (e.g., cefazolin sodium, cephradine, cefaclor, cephapirin sodium, ceffizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), prythronycins, penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., tetracycline hydrochloride, doxycycline hyclate, minocycline hydrochloride, and the like), and the like);

anti-infectives (e. g., GM-CSF);

bronchodilators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents (e.g., ipratropium bromide), xanthines (e.g., aminophylline, dyphylline, metaproterenol sulfate, aminophylline), mast cell stabilizers (e.g., cromolyn sodium), inhalant corticosteroids (e.g., flurisolidebeclomethasone dipropionate, beclomethasone dipropionate monohydrate), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide, and the like);

hormones (e.g., androgens (e.g., danazol, testosterone cypionate, fluoxymesterone, ethyltostosterone, testosterone enanihate, methyltestosterone, fluoxymesterone, testosterone cypionate), estrogens (e.g., estradiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like), thyroid hormones (e.g., levothyroxine sodium) and the like), and the like;

hypoglycemic agents (e.g., human insulin, purified beef insulin, purified pork insulin, glyburide, chlorpropamide, glipizide, tolbutamide, tolazamide, and the like);

hypolipidemic agents (e.g., clofibrate, dextrothyroxine sodium, probucol, lovastatin, niacin, and the like);

proteins (e.g., DNase, alginase, superoxide dismutase, lipase, and the like);

nucleic acids (e.g., sense or anti-sense nucleic acids encoding any therapeutically useful protein, including any of the proteins described herein, and the like);

agents useful for erythropoiesis stimulation (e.g., erythropoietin);

antiulcer/antireflux agents (e.g., famotidine, cimetidine, ranitidine hydrochloride, and the like);

antinauseants/antiemetics (e.g., meclizine hydrochloride, nabilone, prochlorperazine, dimenhydrinate, promethazine hydrochloride, thiethylperazine, scopolamine, and the like);

oil-soluble vitamins (e.g., vitamins A, D, E, K, and the like);

as well as other drugs such as mitotane, visadine, halonitrosoureas, anthrocyclines, ellipticine, and the like.

Other examples of the therapeutic compounds that may be used in the present invention for solubility and bioavailability enhancement are those active ingredients in traditional Chinese medicine, for example, cucurmin and indirubin.

Although the technology described in the current invention is particularly useful for enhancing the bioavailability of poorly water soluble drugs, it can also be used for delivering water soluble drugs for other benefits such as improvement in drug stability, pharmaco-dynamics and pharmaco-kinetics. Examples of water soluble drugs are small molecule drugs, proteins, peptides, antibodies, oligonucleotides, vaccines, and hormones.

As described above, a biocompatible oil capable of solubilizing the therapeutically active compound may be used in the present invention. In one embodiment of the current invention, at least one therapeutically active compound is dissolved in said biocompatible oil to form a homogeneous solution. The weight fraction of the drug in the discontinuous phase, which is defined as the total mass of the drug dissolved divided by the total mass of the discontinuous phase droplets, is between 0.1-90%, preferably between 1-50%.

Alternatively, a biocompatible oil that does not solubilize the therapeutically active compound may also be used as long as the therapeutically active compound can be made into small-sized solid particles and suspended in said biocompatible oil to form a stable suspension. The therapeutically active compound can be suspended in the suspending oil by the use of mechanical forces and surfactants to form a stable drug suspension. In the case the therapeutically active compound is suspended as particles in the biocompatible oil, it is preferred that the particles are small in size, preferably below 10 microns, more preferably below 2 microns, even more preferably below 500 nm. The weight fraction of the drug in the discontinuous phase is between 0.1-90 wt %, preferably between 1-50 wt %.

There are various ways of making therapeutically active compounds into small sized particles, for example, by milling, homogenization, or precipitation techniques. Exemplary methods of making nanoparticulate compositions are described in U.S. Pat. No. 5,145,684 for “Surface Modified Drug Nanoparticles.” Methods of making nanoparticulate compositions are also described in U.S. Pat. No. 5,518,187 for “Method of Grinding Pharmaceutical Substances;” U.S. Pat. No. 5,156,842 for “Liquid Suspension for Oral Administration;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” U.S. Pat. No. 5,470,583 for “Method of Preparing Nanoparticle Compositions Containing Charged Phospholipids to Reduce Aggregation;” and U.S. Pat. No. 7,390,505 for “Nanoparticulate Topiramate Formulations,” all of which are specifically incorporated by reference.

1. Milling to Obtain Nanoparticulate Drug Dispersions

Milling the therapeutically active compound to obtain a nanoparticulate dispersion comprises dispersing therapeutically active compound particles in a liquid dispersion media in which it is poorly soluble, followed by applying mechanical means in the presence of grinding media to reduce the particle size of the therapeutically active compound to the desired effective average particle size. The dispersion media is a biocompatible oil.

The drug particles can be reduced in size in the presence of at least one surface stabilizer. Alternatively, the drug particles can be contacted with one or more surface stabilizers after attrition. Other compounds, such as a diluent, can be added to the drug/surface stabilizer composition during the size reduction process. Dispersions can be manufactured continuously or in a batch mode.

2. Precipitation to Obtain Nanoparticulate Drug Suspensions

Another method of forming the desired nanoparticulate drug composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the drug in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Drug Suspensions

Exemplary homogenization methods of preparing active agent nanoparticulate compositions are described in U.S. Pat. No. 5,510,118, for “Process of Preparing Therapeutic Compositions Containing Nanoparticles.”

Such a method comprises dispersing drug particles in a liquid dispersion media in which the drug is poorly soluble, followed by subjecting the dispersion to homogenization to reduce the particle size of the therapeutic compound to the desired effective average particle size.

The drug particles can be reduced in size in the presence of at least one surface stabilizer. Alternatively, the drug particles can be contacted with one or more surface stabilizers either before or after attrition. Other compounds, such as a diluent, can be added to the drug/surface stabilizer composition before, during, or after the size reduction process. Dispersions can be manufactured continuously or in a batch mode.

The discontinuous phase of the present invention, i.e. the biocompatible oil containing therapeutic compounds dissolved or suspended therein, is dispersed in an aqueous or a non-aqueous media to form microdroplets or nanodroplets by mechanical forces and optionally by the presence of one or a mixture of emulsifiers. The droplets may be generally spherically shaped or may resemble shapes that are not spherically shaped. The drug dissolved or dispersed in the biocompatible oil has a weight fraction of the drug in the discontinuous phase of between 0.1-90 wt %, preferably between 1-50 wt %.

For specially shaped droplets as depicted in FIG. 3A, the size of the droplets can be characterized using the mean value of the diameter of the droplets A in the sample. For non-spherically shaped droplets 9 as depicted in FIG. 3B, i.e., imperfectly spherically shaped droplets, the mean value of the quasi-diameter may be used for the size characterization of the droplets.

The droplets of the discontinuous phase of the present invention may be microdroplets or nanodroplets, having effective average sizes of from 1 nm to 1 mm, preferably from 10 nm to 500 μm.

The continuous phase of the present invention comprises a solid or solid-forming material and optionally an emulsifier. Said solid material may be a pharmaceutically acceptable polymer. Combinations of more than one polymer can be used in the invention. Useful continuous phase polymers which can be employed in the invention include, but are not limited to, known organic and inorganic pharmaceutical excipients. Such excipients include various polymers, low molecular weight oligomers, and natural products. In the present invention, said polymer is preferably a water-soluble polymer. The polymers having number average molecular weights of from 500 to 50 millions, preferably from 2,500 to 5 millions, may be used. Polymers that are preferred in the present invention include but are not limited to polyvinyl alcohol, poly(vinyl acetate), polyvinylpyrrolidone, poly(acrylic acid), poly(acrylic acid) ammonium salt, poly(acrylic acid) sodium salt, polyacrylamide, poly(ethylene oxide), poly(ethylene glycol), poly(hydroxyethyl methacrylate), polyethyleneimine, starch, poly(N-isopropyl acrylamide), cellulose, dextran, gelatin, chitin, chitosan, the copolymers and mixtures thereof.

Representative examples of other useful continuous phase polymers include hydroxypropyl methylcellulose, hydroxypropylcellulose, casein, lecithin (phosphatides), gum acacia, cholesterol, tragacanth, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Specialty Chemicals)); polyethylene glycols (e.g., Carbowaxs 3550° and 934° (Union Carbide)), polyoxyethylene stearates, carboxymethylcellulose calcium, carboxymethylcellulose sodium, methylcellulose, hydroxyethylcellulose, hydroxypropylmethylcellulose phthalate, noncrystalline cellulose, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

As already mentioned, an emulsifier may also be used to assist the formation of the discontinuous phase droplets. The emulsifier is a pharmaceutically acceptable surfactant, which may be a small molecule, oligomer or polymer. It may be nonionic, cationic or anionic. It may be of natural or synthetic origin.

Representative examples of the emulsifier include, but are not limited to, gelatin, casein, lecithin (phosphatides), gum acacia, cholesterol, tragacanth, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, microcrystalline cellulose, magnesium aluminum silicate, triethanolamine, polyvinyl alcohol, polyvinylpyrrolidene (PVP), stearic acid, benzalkonium chloride, calcium stearate, glycerol monostearate, cetostearyl alcohol, cetomacrogol emulsifying wax, and sorbitan esters. Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Other examples of surfactants include tyloxapol, poloxamers such as Pluronic F68, F77, and F108, which are block copolymers of ethylene oxide and propylene oxide, and polyxamines such as Tetronic 908 (also known as Poloxamine 908), which is a tetrafunctional block copolymer derived from sequential addition of propylene oxide and ethylene oxide to ethylenediamine, available from BASF, dextran, lecitin, dialkylesters of sodium sulfosuccinic acid, such as Aerosol OT, which is a dioctyl ester of sodium sulfosuccinic acid, available from American Cyanamid, Duponol P, which is a sodium lauryl sulfate, available from DuPont, Triton X-200, which is an alkyl aryl polyether sulfonate, available from Rohm and Haas, Tween 20 and Tween 80, which are polyoxyethylene sorbitan fatty acid esters, available from ICI Specialty Chemicals; Carbowax 3550 and 934, which are polyethylene glycols available from Union Carbide; Crodesta F-110, which is a mixture of sucrose stearate and sucrose distearate, available from Croda Inc., Crodesta SL-40, which is available from Croda, Inc., and SA9OHCO, which is C₁₈H₃₇—CH₂(CON(CH₃)CH₂(CHOH)₄CH₂OH)₂, decanoyl-N-methylglucamide; n-decyl.beta-D-glucsopyranoside; n-decyl.beta-D-maltopyranoside; n-dodecyl.beta-D-glucopyranoside; n-dodecylβ-D-maltoside; heptanoyl-N-methylglucamide; n-heptyl-β-D-glucopyranoside; n-heptylβ-D-thioglucoside; n-hexylβ-D-glucopyranoside; nonanoyl-N-methylglucamide; n-noylβ-D-glucopyranoside; octanoyl-N-methylglucamide; n-octyl-β-D-glucopyranoside; octyl-β-D-thioglucopyranoside; and the like.

Generally, there are two preferred methods which are used in the present invention to prepare the two-phase drug delivery medium. In both cases, the discontinuous fluid is preferably a polar solvent for dissolving the continuous phase polymer. Said polar solvent is exemplified by, but not limited to, water, methyl alcohol, ethyl alcohol, isopropyl alcohol, acetone, ethyl acetate, tetrahydrofuran, and the mixture thereof. Water is highly preferred in the present invention for dissolving the continuous phase materials.

The first method used in the present invention to prepare the bioavailability-enhancing drug delivery formulation comprises the following steps:
1) Preparing the drug-containing discontinuous phase by either dissolving or suspending the drug in the biocompatible oil, as described above; the drug dissolved or dispersed in the biocompatible oil has a weight fraction of the drug in the discontinuous phase of between 0.1-90 wt %, preferably between 1-50 wt %;
2) Dissolving the emulsifier in a polar solvent to form an emulsifier solution. Water is highly preferred as said polar solvent. Heat may be used to facilitate the dissolution of the emulsifier. The concentration of the emulsifier solution is preferably from 0 to 20 wt %, more preferably from 0.001 to 10 wt %;
3) Dissolving the continuous phase polymer in the polar solvent described in Step 2) to form a continuous phase solution with a concentration of from 0.1 to 99 wt %, preferably from 1 to 50 wt %, more preferably from 5 to 30 wt %;
4) Mixing the discontinuous phase prepared in Step 1) with the emulsifier solution prepared in Step 2) to form the suspension of the discontinuous phase droplets in the polar solvent. Heat and mechanical agitation (for example, stirring) may be used to facilitate the formation of the oil droplets. The speed of said stirring may be 10-100,000 rpm, preferably 100-10,000 rpm. A homogenizing device such as a rotor stator, homogenizer or micro-fluidizer can be further used to facilitate the formation of the microdroplets or nanodroplets of the discontinuous phase. The combined effect of the use of the emulsifier and mechanical agitation results in a droplet suspension with droplet size ranging from 1 nm to 1 mm, preferably from 10 nm to 500 μm.
5) Mixing the continuous phase polymer solution prepared in Step 3) with the droplet suspension of Step 4). The ratio of the continuous phase to the discontinuous phase is from 0.01 to 100, preferably 0.1 to 50, more preferably from 1 to 10. After mixing, the stirring speed and temperature are maintained for from 0 to 24 hours, preferably from 5 minutes to 5 hours. The stirring speed is then preferably reduced to 10-5,000 rpm, preferably 50-1,000 rpm. The stirring is continued for 1-240 hours, preferably 5-100 hours. The temperature is preferably maintained at from −10 to 95° C., more preferably from 10 to 50° C.
6) The drug delivery medium obtained in Step 5) may be used directly as a drug formulation or be incorporated into capsules. Otherwise the polar solvent in the continuous phase can be removed by evaporation or other drying methods so that the entire system becomes semi-solid or solid for the subsequent dosage form fabrication; for example, by drying and grinding them into particles before tablet preparation, or transformed into granules, pellets, and powders for dry filled capsules. Various methods may be used in the process, for example, melt granulation, adsorption on solid support, spray cooling, melt extrusion/spheronization, freeze-drying and spray drying.

The second method used in the present invention to prepare the bioavailability-enhancing drug delivery formulation comprises the following steps:
A. Preparing the drug-containing discontinuous phase by either dissolving or suspending the drug in the biocompatible oil, as described above; the drug dissolved or dispersed in the biocompatible oil has a weight fraction of the drug in the discontinuous phase of between 0.1-90 wt %, preferably between 1-50 wt %;
B. Preparing a solution comprising an emulsifier and a continuous polymer described as above in the polar solvent. The concentration of the emulsifier solution is preferably from 0 to 20 wt %, more preferably from 0.001 to 10 wt %. The concentration of the continuous polymer is from 0.1 to 99 wt %, preferably from 1 to 50 wt %, more preferably from 5 to 30 wt %. Water is highly preferred solvent in the present invention. It is noted that in this case the emulsifier and the discontinuous polymer may or may not be the same substance; therefore, the use of either component may be optional;
C. Mixing the discontinuous phase prepared in Step A with the solution prepared in Step B to form the suspension of the discontinuous phase droplets. The ratio of the continuous phase to the discontinuous phase is from 0.01 to 100, preferably 0.1 to 50, more preferably from 1 to 10. Heat and mechanical agitation (for example, stirring) may be used to facilitate the formation of the oil droplets. The speed of said stirring may be 10-100,000 rpm, preferably 100-10,000 rpm. A homogenizing device such as a rotor stator, homogenizer or micro-fluidizer can be further used to facilitate the formation and dispersing of the microdroplets or nanodroplets of the discontinuous phase. The droplet suspension is formed with droplet size ranging from 1 nm to 1 mm, preferably from 10 nm to 500 μm;
D. The stirring speed and temperature are maintained for from 0 to 24 hours, preferably from 5 minutes to 5 hours. The stirring speed is then preferably reduced to 10-5,000 rpm, preferably 50-1,000 rpm. The stirring is continued for 1-240 hours, preferably 5-100 hours. The temperature is preferably maintained at from −10 to 95° C., more preferably from 10 to 50° C.
E. The drug delivery medium obtained in Step D may be used directly as a drug formulation or be incorporated into capsules. Otherwise the polar solvent in the continuous phase can be removed by evaporation or other drying methods so that the entire system becomes semi-solid or solid for the subsequent dosage form fabrication; for example, by drying and grinding them into particles before tablet preparation, or transformed into granules, pellets, and powders for dry filled capsules. Various methods may be used in the process, for example, melt granulation, adsorption on solid support, spray cooling, melt extrusion/spheronization, freeze-drying and spray drying.

The drug delivery medium obtained in Step 6) or Step E above may also be processed into a film by coating the wet formulation onto an appropriate substrate before drying and forming a film. For example, bar coating or roll-to-roll coating may be used for the generation of the film. The resulting film may be used as a drug delivery composition for the treatment of mammals.

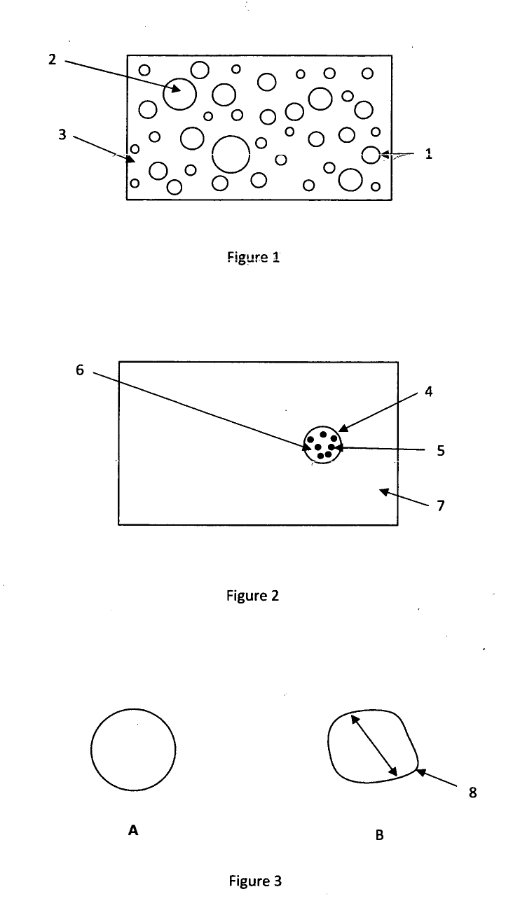

The drug delivery medium of the present invention is illustrated in FIGS. 1 and 2. FIG. 1 depicts the drug delivery medium comprising the discontinuous droplets 1 wherein the drug is dissolved in the discontinuous fluid to form the drug-oil solution 2, and the continuous phase 1.

FIG. 2 depicts the drug delivery medium comprising the discontinuous droplets 4 wherein the drug particles 5 are suspended in the discontinuous fluid 6 to form the drug-oil suspension, and the continuous phase 7.

Optionally, drugs may also be dispersed in the continuous phase of the present invention.

The following Examples are now given, though by way of illustration only, to show details of particularly preferred reagents, conditions and techniques used in the present drug delivery medium and process for its preparation.

### EXAMPLE 1

In a jacketed flask connected to a circulating water bath, 0.5 g food and drug grade gelatin was dissolved in 7.5 ml distilled water at 42° C. Separately 600 mg of Paclitaxel was dissolved in 10 ml triacetin with minimal heating at 35° C. The gelatin solution was stirred mechanically at 400 rpm while the Paclitaxel solution was added to it. After 10 minutes, 30 ml of 10% aqueous solution of Providone K-30 was added to the flask. The stirring speed was then reduced to 250 rpm and the temperature lowered to 25° C. The stirring was continued for 24 hours.

### EXAMPLE 2

In a jacketed flask connected to a circulating water bath, 3 g food and drug grade gelatin was dissolved in 30 ml distilled water at 42° C. Separately 600 mg of Paclitaxel was dissolved in 10 ml triacetin with minimal heating at 35° C. The gelatin solution was stirred mechanically at 350 rpm while the Paclitaxel solution was added to it. After 30 minutes, the temperature was lowered to 25° C., whereas the stirring was continued for 24 hours.

### EXAMPLE 3

In a jacketed flask connected to a circulating water bath, 0.5 g food and drug grade gelatin and 3 g Providone K-30 were dissolved in 30 ml distilled water at 42° C. Separately 600 mg of Paclitaxel was dissolved in 10 ml triacetin with minimal heating at 35° C. The gelatin-Providone solution was stirred mechanically at 350 rpm while the Paclitaxel solution was added to it. After 30 minutes, the temperature was lowered to 25° C., whereas the stirring was continued for 24 hours.

### EXAMPLE 4

The mixture containing Paclitaxel, gelatin, triacetin and water prepared in EXAMPLE 1 was freeze dried.

### EXAMPLE 5

The mixture containing Paclitaxel, gelatin, triacetin and water prepared in EXAMPLE 1 was spray dried.

### EXAMPLE 6

The mixture containing Paclitaxel, gelatin, triacetin and water prepared in EXAMPLE 1 was coated on a poly(ethylene terephthalate) film using a bar coater. The coating was allowed to dry in the air and the resulting film was collected. The thickness of the film was found to be 30 microns.

### EXAMPLE 7

In a jacketed flask connected to a circulating water bath, 0.5 g food and drug grade gelatin was dissolved in 7.5 ml distilled water at 42° C. Separately, 2 g sorbitol, 0.5 g citric acid, 2 g Tenox GT-1, 3 g Aerosil R972, and 400 mg Erhthromycin ethyl succinate were ball milled with 8 g Soya Oil U.S.P. for 5 hours. The resulting suspension was added to the gelatin solution with mechanical stirring at 450 rpm. After 10 minutes, 30 ml of 10% aqueous solution of Providone K-30 was added to the flask. The stirring speed was then reduced to 300 rpm and the temperature lowered to 25° C. The stirring was continued for 24 hours.

What is claimed is:

1. A two-phase drug delivery medium comprising a discontinuous phase and a solid continuous phase, the discontinuous phase comprising a plurality of droplets, each of which comprises a fluid and at least one drug dissolved or suspended within the fluid, and the continuous phase surrounding and encapsulating the discontinuous phase.

2. The discontinuous phase of claim 1, wherein the fluid is a pharmaceutically acceptable oil.

3. The discontinuous phase of claim 1, wherein the droplets are microdroplets having diameters or quasi-diameters from 1 to 1,000 microns.

4. The discontinuous phase of claim 1, wherein the droplets are nanodroplets having diameters or quasi-diameters from 1 to 1,000 nanometers.

5. The discontinuous phase of claim 1, wherein the drug is dissolved in the fluid.

6. The discontinuous phase of claim 1, wherein the drug is suspended as particles in the fluid with particles sizes ranging from 1 nanometer to 1,000 micrometers.

7. The discontinuous phase of claim 2, wherein the pharmaceutically acceptable oil is selected from the group containing almond oil, castor oil, corn coli, cottonseed oil, mineral oil, medium-chain or long-chain tri-, di- and mono-glycerides, triacetin, diacetin, tocopherol, oliver oil, peanut oil, sesame oil, soybean oil, sunflower oil, and the mixture thereof.

8. The discontinuous phase of claim 6, wherein the drug suspension further comprises at least one stabilizer preferably absorbed on the surface of the drug particles.

9. The drug delivery medium of claim 1, wherein the continuous phase comprises at least one pharmaceutically acceptable polymer.

10. The continuous phase of claim 9, wherein the pharmaceutically acceptable polymer is a hydrophilic polymer and is selected from the group containing polyvinyl alcohol, poly(vinyl acetate), polyvinylpyrrolidone, poly(ethylene glycol), poly(acrylic acid), poly(acrylic acid) ammonium salt, poly(acrylic acid) sodium salt, polyacrylamide, poly(ethylene oxide), poly(hydroxyethyl methacrylate), polyethyleneimine, starch, poly(N-isopropyl acrylamide), cellulose, cellulous derivatives, dextran, gelatin, chitin, chitosan, the copolymers and mixtures thereof.

11. The continuous phase of claim 9 further comprises an emulsifier.

12. The continuous phase of claim 11, wherein the emulsifier is gelatin.

13. The drug delivery medium of claim 1 wherein the drug to be delivered is a poorly water-soluble drug.

14. A process for producing a two-phase drug delivery medium, this process comprising:
providing a liquid medium comprising a solid-forming material;
dispersing in the liquid medium a plurality of droplets, each of which comprises a fluid and at least one drug dissolved or suspended within the fluid, thereby forming a droplet-containing liquid medium; and
subjecting the droplet-containing liquid medium to conditions effective to cause the solid-forming material therein to form a solid or semi-solid, and thereby producing a two-phase drug delivery medium in which the solid-forming material forms a solid or semi-solid continuous phase surrounding and encapsulating the droplets, which form the discontinuous phase of the drug delivery medium.
